(19) United States
(12) Patent Application Publication
(10) Pub. No.: US 2010/0036211 A1
(43) Pub. Date: Feb. 11, 2010

(54) SYSTEMS AND METHODS FOR MEASURING PHYSIOLOGICAL PARAMETERS OF A BODY

(75) Inventors: George S. La Rue, Pullman, WA (US); David Rector, Pullman, WA (US); Deuk Heo, Pullman, WA (US)

Correspondence Address: PERKINS COIE LLP;PATENT-SEA, P.O. BOX 1247, SEATTLE, WA, 98111-1247, US

(73) Assignee: WASHINGTON STATE UNIVERSITY, Pullman, WA (US)

(21) Appl. No.: 12/513,839

(22) PCT Filed: Nov. 7, 2007
(86) PCT No.: PCT/US2007/083926

§ 371 Date: May 6, 2009

Related U.S. Application Data
(60) Provisional application No. 60/857,604, filed on Nov. 7, 2006.

(51) Int. Cl. A61B 5/00

(52) U.S. Cl. 600/301

(57) ABSTRACT

Systems and methods for measuring physiological parameters of a body are described. In some embodiments, the system employs a physiological monitoring device capable of measuring, amplifying, digitizing and transmitting events ranging in frequency from 0 (DC) to 10 kHz.

### CROSS REFERENCE(S) TO RELATED APPLICATIONS

This application claims the benefit of U.S. Provisional Patent Application No. 60/857,604, filed on Nov. 7, 2006, entitled SYSTEMS AND METHODS FOR MEASURING PHYSIOLOGICAL PARAMETERS OF A BODY, which is hereby incorporated by reference in its entirety.

### TECHNICAL FIELD

The present disclosure is related to systems and methods for measuring physiological parameters of a body.

### BACKGROUND

The impact of sleep loss and sleep disorders on the health, social and economic well being of America is enormous. (Yet our knowledge about the control and function of sleep remain severely limited, based largely on studies where subjects are tethered, with significant behavioral side-effects. Thus, compact, implantable recording systems have become a major factor in sleep studies, especially in small transgenic mouse models and long term monitoring where tethering is not practical for many channels of electrophysiology. Existing telemetry systems are severely limited in the amount of information they can gather, and are not conducive for most studies. As a result, researchers have typically gathered data by burdening subjects with heavy wire tethers. The number of wires required can be reduced by amplifying multiplexed signals at the signal source. However, signal processing circuitry currently available is not compact. As a result, these approaches are impractical for use in small animal studies or studies where long term monitoring of any size subject is necessary.

A number of studies have started to take a more detailed look at localized EEG across different brain regions during sleep/wake states. From such studies, it is clear that different brain regions exhibit distinct cortical EEG patterns, even during the same global state. Also, slow wave sleep depth can also be use dependent. The underlying mechanisms that generate the whole animal state and region specific EEG may be different, and a better understanding of these regional differences may explain how sleep is generated, leading to insights into the function of sleep. Several investigators (consider sleep a distributed process, beginning at the cellular level. Built primarily from a biochemical point of view, the distributed theory of sleep regulation makes predictions concerning the electrophysiological behavior of individual neural groups. Additionally, recent developments in high density electrode arrays for cortical mapping and fine-wire recordings from freely moving animals underscore a major need for many channels in neural network analysis and especially in behavioral studies. Thus, the development of surface mapping electrode arrays coupled to the appropriate signal processing and transmission systems are a necessary extension of existing technologies.

Traditional sleep recordings sample 4 to 6 channels at 200 Hz. Typically, EMG, EEG, EKG and respiration are usually adequate for assessing the state of the animal, but are limited by the amount of wire the animal can carry and the acquisition capacity of the data system. However, new approaches to study the function and control of sleep state involve mapping either large brain regions with high temporal and spatial resolution or unit firing patterns from a large number of cells. Increasingly, sleep studies require many more channels and much higher sample rates to assess patterns of unit activity across a large number of cells simultaneously, or high density electrode arrays for EEG mapping. In order for such a system to be feasible the size must be small, and ideally, use telemetry for untethered recording.

Typical electrophysiological scalp recordings utilize multiple electrode positions, however brain mapping using external electrodes have poor spatial resolution for source localization. Unfortunately, better localization is achieved by placing the electrodes closer to the tissue: either directly in contact with the tissue surface, or penetrating into the tissue. For example, neurosurgery often requires detailed maps of brain surface function before resections of damaged or tumor tissue. For this application, surgeons use surface electrode arrays embedded in flexible plastic sheets placed on the brain surface. In order to access large brain surface regions, significant portions of the skull must be removed, introducing additional trauma to the tissue. A number of electrode arrays have been developed for both acute and chronic/awake preparations. However, all of these electrodes are either penetrating unit electrodes, or require large portions of the skull to be removed in order to expose enough of the brain surface for mapping. Large skull openings, however, are difficult to maintain chronically.

For many chronic animal preparations, long cables, tethering, and rotational movement through a commutator are adequate solutions for electrophysiological recording. However, investigators require better signal-to-noise, especially during excessive movement, or when electrodes have very high impedance, such as within unit electrodes. In these cases, it may necessary to place the amplifiers on the animal to provide signal amplification and possibly some filtering before being transmitted by the long cable and commutator. Solutions range from single FET transistors, to a miniature circuit board with several amplifiers embedded on the animal's head (e.g. http://www.neuralynx.com). Some investigators have started to use custom designed amplifier chips with several channels, and some additional functionality. All of the solutions for many-channel electrophysiology still require significant cabling, and have a relatively large size.

The advancement of complementary metal oxide semiconductor (CMOS) integrated circuit (IC) technology has enabled rapid improvement in the performance and density of electronics. The high density and good performance at low power of CMOS has enabled affordable portable applications such as cell phones, wireless networking, wireless computer mice, etc. Within the art of electrophysiological monitoring, a wireless implantable system has been demonstrated. It uses an inductively coupled RF telemetry link to supply power and to passively transmit data from the implanted device. It only digitizes two 3.1 kHz bandwidth channels at a time with 8-bit accuracy.

Commercial remote sensors are available from Transoma Medical to provide monitoring in mice of only one biopotential (EEG, ECG or EMG) channel limited to a 100 Hz frequency response. There are other commercial remote telemetry products from Data Integrated Scientific Systems with up to 6 biopotential channels but these multichannel devices are too large for mice. There are several other multi-channel preamplifiers commercially available, but these do not provide analog-to-digital conversion on the IC. For example, Plexon offers preamplifiers on small printed circuit boards with 8, 16 and 32 channels and gains of 1 and 20 (www.plexoninc.com). Nicolelis has published results on 2 multi-channel integrated circuits for neural recording. These are small-volume 16-channel devices; one with a gain of 2 and the other has a high-pass filter and selectable gains of 250-500.

TABLE 1
Comparison of Published Preamplifiers

| | | | Noise | | Power per | | Data | |
|---|---|---|---|---|---|---|---|---|
| Project | Channels | Gain | (uVrms) | Bandwidth | Channel | A/D | Rate | Comments |
| Obeid | 16 | 2 or | 4.4 | 400 Hz to | 0.95 to 1.4 mW | N/A | (Analog) | Preamplifiers only |
| (2003) | | 250/500 | | 22 kHz | | | | |
| Olsson | 8 | 100 | ? | 100 Hz to | 0.1 mW | N/A | (Analog) | 64 to 8 channel |
| (2002) | (64 sites) | | | 10 kHz | | | | selector in front end |
| Akin | 2 | 100 | ? | 100 Hz to | 5 mW | 8 | 100 | Wireless digital data |
| (1998) | | | | 3.1 kHz | | bits | Kbits/s | Inductively powered |
| Transoma | 1 to 6 | ? | ? | DC to | ? | N/A | (Analog) | Wireless analog data |
| Medical | | | | 100 Hz | | | | Battery powered |

Few commercial sources offer telemetric systems for recording physiology from small animals (e.g., www.datasci.com), and adequate physiological signal telemetry from humans has only recently been implemented (e.g., www.monitoring.welchallyn.com/products/wireless). The performance of such systems, however, is still poor, providing only a few channels at low bandwidth. Recent advances in digital communications (Bluetooth and other wireless networking, digital cell phones, and satellite communications) has significantly increased the ability to transfer wireless information with small, cheap, and low power devices.

To implement a wireless link in telemetry systems, different methods have been applied. Investigators at the University of Michigan used a passive wireless link using inductive coupling between the primary and secondary coils for both power and data transfer. Their early designs used amplitude modulation (AM) to send data to the base station with a 100 Kbit/s data rate. Their recent wireless link is targeted to use a 4˜20 MHz carrier frequency to obtain the baseband amplitude modulated carrier envelope and designed to extract a data stream up to 2 Mbit/sec from the frequency shift keying (FSK) modulated RF carrier. They reported that the prototype chip and its individual blocks have been tested up to 200 Kbit/s and were fully functional.

Several companies have developed simple AM radio links to send low-data-rate signal to the base station. Transoma Medical uses a channel limited to 100 Hz and J&J Engineering provides wearable physiology monitoring RF telemetry system for humans. Further, the Academic Medical Center in the Netherlands uses a wireless infrared link for a 16-channel EEG telemetry system. The system uses a sample rate of 1 kHz per channel and an accuracy of 12-bits, and was designed to handle up to 192 Kbit/sec data stream. The infrared transmission has a disadvantage of the low efficiency of optical transmitter and receiver components. In addition, it requires an uninterrupted light path between the transmitter and receiver. Table 2 summarizes recent results for wireless links of telemetry systems.

TABLE 1
Specifications of previous wireless links of telemetry systems

| Project | Data Rate | | Integration | Frequency Band |
|---|---|---|---|---|
| M. Modarreszadeh (1997) | 40 | Kbit/s | Multi-chip module | 902-928 MHz FSK modulation |
| K. D. Wise (2002) | 100 | Kbit/s | Multi-chip module | 4 MHz AM modulation |
| S. Takeuchi (2000) | 256 | Kbit/s | Multi-chip module | 80-90 MHz Analog FM modulation |
| M. Ghovanloo (2003) | 2 | Mbit/s | Multi-chip module | 88-108 MHz FSK modulation |
| H. Yu (2004) | 1.6 | Mbits/s | Multi-chip module | 49 MHz ASK modulation |

| | | | |
|---|---|---|---|
| P. Mohseni (2003) | (Analog) | Multi-chip module | 88-108 MHz Analog FM modulation |
| P. Irazoqui-Pastor (2004) | (Analog) | Single Chip (Single channel) | 3.2 GHz FM |
| R. Simons (2004) | (Analog) | Single Chip | 230-670 MHz |

In order for an untethered animal (which may or may not be human) to be effectively recorded from, the animal also be instrumented with a power supply that can last for the duration of the experiment. Commercially available devices use implantable batteries with 100 hours or more working life times. Magnetically activated switches are used to activate or deactivate the unit to preserve battery life. For very long experiments, however, an unlimited amount of power may be required, especially since a device with up to many (>16) channels will require significantly more power than existing devices.

### BRIEF DESCRIPTION OF THE DRAWINGS

FIG. 1A is a block diagram illustrating a pathway of components for measuring physiological parameters of a body.

FIG. 1B is a block diagram illustrating a system for measuring physiological parameters of a body.

FIG. 2A is a schematic diagram illustrating input DC offset cancellation with unequal source resistances.

FIG. 2B is a schematic diagram illustrating input DC offset cancellation with body bias.

FIG. 2C is a schematic diagram illustrating input DC offset cancellation with body bias and chopping.

FIG. 3 is a schematic diagram illustrating an analog to digital converter (ADC).

FIG. 4 is a diagram of a power generation component.

FIG. 5 is a block diagram illustrating a data system for measuring physiological parameters of a body.

FIG. 6 is a chart illustrating an evoked response from a body.

FIG. 7 is a diagram illustrating an electrode array.

FIG. 8 is a block diagram illustrating a chip fabrication process for a physiological signal measuring device.

FIG. 9 is a chart illustrating an evoked response from a body.

FIG. 10 is a schematic diagram of an amplifier and digitizer system.

FIG. 11 is a diagram of a preamplifier chip.

FIG. 12 is a diagram of components of a wireless transmission system.

FIG. 13 is a chart illustrating test results from various devices described herein.

FIGS. 14-15 are charts illustrating various test results.

FIG. 16 is a chart illustrating transmissions from an OOK transmitter.

FIG. 17 is a plot illustrating power transfer efficiency of a recording cage for a subject.

### DETAILED DESCRIPTION

There is a need for physiological monitoring equipment that combines a unique and technologically challenging set of attributes. One system includes the ability to generate a detailed map of the activity for a highly localized physical area and cover a broad range of events spanning in frequency from 0 Hz (DC) to ˜10 kHz.

A physiological monitoring device capable of measuring, amplifying, digitizing and transmitting events ranging in frequency from 0 (DC) to 10 kHz is described herein. Many design elements have been incorporated into the various components to achieve such a wide dynamic range without compromising the quality of the data. The device is composed of at least one contact (e.g., a detection element) which contains at least two spatially separated channels. An amplifier and digitizer are electrically connected to the contact and a data transmitter. Each data transmitter is electrically connected to at least one amplifier/digitizer unit. All of the electrodes, amplifier/digitizers and transmitters can be powered by at least one power source. One implementation combines a single amplifier, digitizer, transmission unit, and power source as a system on chip (SoC). Of course, implementations wherein the components act as isolated units can be used.

The implementation described herein demonstrates the ability of the device to record and transmit data associated with electrophysiological measurements on the cortical tissue of mice, however in other embodiments the device may measure any number of signals at any point or points on a subject (that may or may not be human). Moreover, various implementations are considered wherein multiple detection elements are positioned at distinct remote points on a subject. Under this implementation each detection element is connected to an amplifier/digitizer unit that is electrically connected to a wireless transmission unit. Additional implementations wherein multiple detection elements are electrically connected to a single amplifier/digitizer unit that is electrically connected to a wireless transmission unit and/or where a plurality of single detection element-amplifier/digitizer units are connected to a single transmission unit.

### Overview

A physiological monitoring device capable of measuring, amplifying, digitizing and transmitting events ranging in frequency from 0 (DC) to 10 kHz is described herein. There may be specific requirements for devices that provide for the ability to sample, filter and/or amplify analogue data simultaneously from a plurality of channels while providing a wide dynamic range (high bit resolution), high signal to noise, and sample at a high frequency. In one implementation, the device can be secured to a subject and transmit the recorded data wirelessly, and the system may be designed to minimize power consumption and maximize lifetime.

First, a detection element composed of a plurality channels (or a plurality of detection elements each with at least one channel) capable of providing spatial resolution can be included. In certain implementations electrophysiological activity of a subject can be measured with a single electrode and/or an array of finely spaced channels (e.g. electrodes).

Electrodes may have many different compositions and locations including but not limited to: gold arrays, silver/silver chloride, platinum, tungsten or stainless steel, either on top of the skin, under the skin, under the skull and on top of the dura, or under the dura. Other detection element compositions may be utilized either in parallel or in combination with an electrode. Examples include but are not limited to: wire or piezo strain gages, thermistors or thermocouples, coils sensitive to magnetic field, photodiodes, pneumotachometers, chemical sensors, glass electrodes, ultra-miniature wires. Depending on the specific electrophysiological parameter of interest the detection element may be placed on the subject at a single or various points. In some implementations the detection element can be secured to the surface (e.g. skin) of the subject while in other applications the detection element may be inserted into the subject (e.g. placed onto the cortical surface). To enable applications wherein the detection element is placed inside a subject the implementation can utilize a flat, flexible detection element composed of biologically innocuous materials.

The detection element may be comprised of an array of channels covering an area. The area covered may be comprised of a circle, square, rectangle, triangle or any other quasi two-dimensional shape. In some implementations the detection element may comprise a cap or other garment which is worn by a subject. Fabrication techniques may presently limit both the number of channels, the composition and the subsequent size of the detection element, however any number of channels positioned within a detection element comprised of any shape or size limited only by the density that may be provided by modern methods for the production of such elements.

For implementations wherein the electrical potential is the variable of interest; detection elements may be composed of any number of conductive materials housed within an insulating medium. To retain the small and flexible properties a thin film of a conductive material (e.g. gold) can be deposited onto a suitable prepared substrate material (e.g. polyimide). The substrate material may or may not be pre-treated to enable patterning of the thin film. Electrodes and interconnects can then be patterned on the surface through any number of techniques available to those skilled in the art. A particular implementation may involve utilizing photolithography to generate the pattern on the surface. Once the conductive material has been patterned, a thin film of insulating material can be deposited and through selectively etching the resulting surfaces the electrode(s) surfaces can be made available for electrically sensing the environment. Many potential derivative methods for the manufacture of small, flexible electrodes exist and one skilled in the art would have many options for the production of such components.

### The Signal Processing Pathway

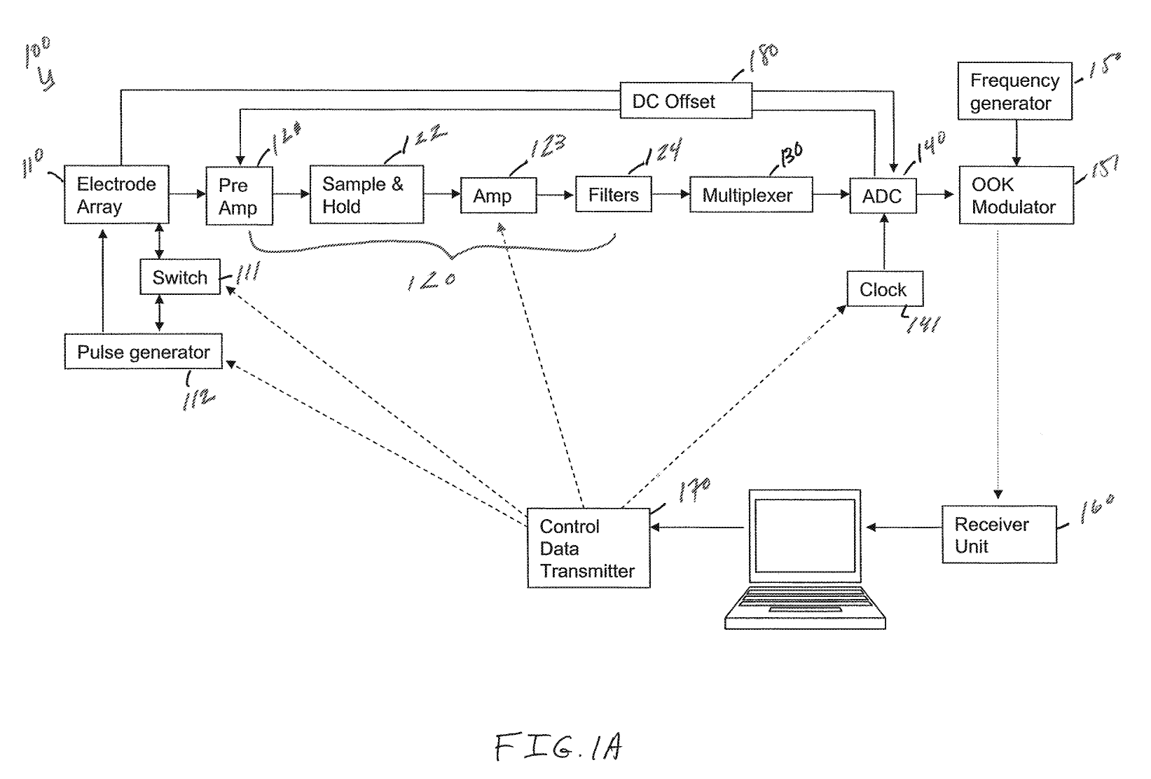

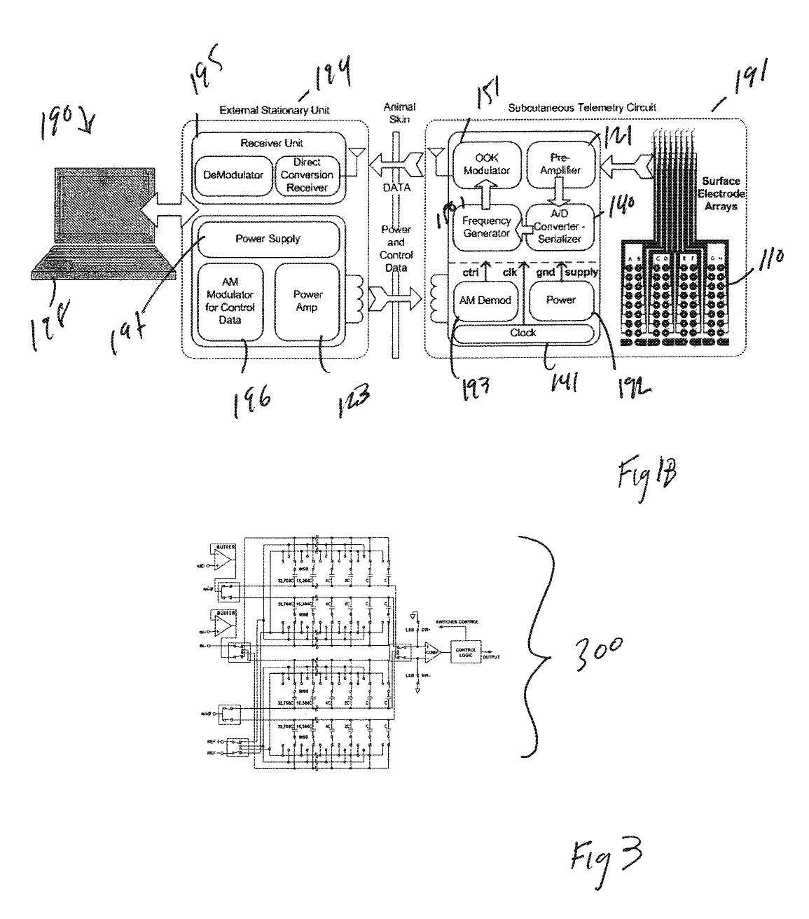

The signal processing circuitry employed in the device described herein can be specifically tailored to maximize the frequency range and minimize the power requirements. A schematic overview of the signal processing pathway is provided in FIGS. 1A and 1B. For example, FIG. 1A is a block diagram illustrating components within the signal processing pathway100 employed by the system. The pathway 100 includes a small and flexible detection element 110 (e.g., an electrode array) composed of a plurality of channels (or a plurality of detection elements each with multiple channels) capable of providing high spatial resolution. For the purposes of this description “detection element” shall mean a detector or detector array comprising a plurality of channels wherein each channel measures a physiological parameter at a location. The channel may measure variables including but not limited to: EEG, EKG, blood pressure, temperature, blood volume, magnetoencephalographic signals (MEG), optical signals, proprioceptive signals, air flow, chemical concentrations, membrane potentials and/or muscle activity. The specific nature of the detection element can be tailored to the target variable, more generally any physical parameter that can be detected and converted into an electrical signal could be measured by this device. In other embodiments, the device may also apply a stimulus, such as an electrical charge, to the body. For example, the detection elements may include electrodes for applying a charge to the body in lieu of or in addition to detecting a physiological parameter of the body.

The pathway also includes a sensitive preamplifier/amplifier system 120 capable of amplifying and filtering signals originating from the detection element(s) in parallel. The preamplifier/amplifier system 120 may include a preamplifier 121, a sampling component 122, an amplifier 123, and/or a filtering component 124. Such a system may have a great degree of flexibility in its ability to process a large range of signals that vary both in frequency and intensity by using some or all of the components 121-124.

Depending on the implementation, the signal may be sent through a multiplexer 130 to serially transfer the data to an analogue to digital converter (ADC) 140 with sufficient resolution (e.g., >12 bit) to capture a wide dynamic range. The data is digitized in such a way to preserve the original temporal attributes (e.g., DC-10 kHz) of and attach any corrections applied to each channel. The device, in some implementations then transmits the data wirelessly, thereby removing the limitations associated with tethered devices. For example, the pathway100 may transmit a digitized signal to a receiver unit 160 using a frequency generator 150 and modulator 151 to generate a modulated transmission frequency to send the digitized data. In some cases, the wireless transmission rate may be sufficient to sustain a data sampling rate of at least twice the highest frequency of interest in order to facilitate recording of the high frequency events. The system may also employ wired applications, wherein the detection/processing component is wired to the data logger.

The system may employ a control data transmitter 170 to monitor and control what components are employed by the pathway100 when conditioning a data signal via the pathway 100. For example, the control data transmitter 170 may initiate operation of a pulse generator 112 and/or a switch 111 in communication with the electrode array 110 to cause the electrode array 110 to apply a stimulus to a body. Additionally, the transmitter may adjust the amplification of a measured signal, may control a clocking mechanism to reduce clock jitter (that is, error in the signal), and so on.

A block diagram of a monitoring system 190 used to implement the pathway 100 is shown in FIG. 1B. For example, a telemetry circuit 191 includes the electrode array 110, a preamplifier 121, an ADC 140, a frequency generator 150, an OOK modulator 151, and a power component 192 and demodulator 193. The telemetry circuit, such as a subcutaneous unit, detects and/or measures parameters from a body and transmits signals carrying data related to the measured parameters to an external unit 194. The external unit 194 may include a reception component 195 (such as a DC receiver and a signal demodulator), a power amplifier 123, a modulator 196 that modulates control data to be sent back to the telemetry unit 191, and a power supply 197 that supplies sufficient power to sustain the device operation during long term sleep studies. The external unit 197 may also include or communicate with a data logger device 198 capable of receiving, indexing and displaying in real time the information transmitted by the data processor. Of course, the system 190 can have other configurations.

Thus, the system 190 may detect parameters from a body using a detection element 110, amplify a signal using the preamplifier 121, filter the signal, digitize the signal, and send the digitized signal to a transmitter, which can then send control data back to a device that includes the detection element 110.

For each channel within a detection element a location specific signal is continuously monitored by an offset component 180. The result is a signal representative of a voltage derived from the difference between the signal intensity present at the channel and that present at a second reference channel. The signal from each channel is then fed into a low gain, high impedance pre-amplifier 120. Depending on the magnitude of the signal, an offset or correction factor may be applied to the signal to ensure the signal falls within a pre-defined voltage range. This correction factor is generated when the final digitized value of the output signal from the analogue to digital converter (ADC) 140 comes close to the upper or lower limit of the digitizer range. The output from the preamplification stage is fed into an amplifier 123 which applies a user-defined gain. The resulting signal is then sent through a low pass filter 124 to prevent aliasing of the digitized data. In implementations where multiple channels are simultaneously being processed, all of the signal input channels can be sampled simultaneously, then sent through a multiplexer 130 which serially transfers the signal from each channel to a ADC. Within the ADC, the signal is digitized and bundled with information relating to any offset that has been applied to the channel. In implementations where wireless transmission is utilized, one method includes using a frequency generator 150 to produce an Industrial, Science and Medical (ISM) band frequency which is then modulated with On-Off Keying (OOK) 151 to generate a carrier wave that can then be transmitted utilizing an RF antenna. Other alternatives include the utilization of off-the-shelf wireless technologies (e.g. 802.11, Bluetooth). In implementations of these methods the signal processing may be electrically connected to a transmitter device capable of transferring data wirelessly to a remote data storage device. The communication of data may occur directly or may involve a relay point (e.g. a short distance transmission from a transmitter affixed to a subject to a wireless router which subsequently transmits the data online to a remote location).

For wireless applications where the processing and transmission are present on a single chip, each element may not only require limited space, but also may have low power demands and the ability to process and transmit data at rate sufficient to accurately record the higher frequency events while retaining the ability to record DC data. While the implementation described herein has these attributes, other embodiments may have different attributes for each component of the signal processing and transmission system.

In some of the examples provided herein, the pre-amplifier 121 utilizes a low gain high impedance stage with DC offset control to cancel slow fluctuations within the signal. Typical preamplifier systems use a high pass filter to remove DC offsets and slow fluctuations, but in many situations, the DC level and slow signals contain important information that is lost with high pass filtering. Several embodiments of the system described herein do not use a high pass filter, rather, they dynamically adjusts a DC offset control signal to keep the final output signal within an appropriate range for the digitizer. Several embodiments of the invention contemplate the use of high pass filters in implementations wherein slow fluctuations are not of interest. When the digitizer output values approach the upper or lower limits of its range, then it sends a trigger to the offset control circuitry to lower or raise the DC offset on the input to the channel. Since the system is keeping track of the absolute offset control levels, they can be combined with the final ADC output word to represent the digitized value with large dynamic range.

A high gain stage follows the preamplifier such that a user can determine the gain setting for any given channel. A standard gain of 100 can be used because many applications fall within this range. However, to record signals with large and fast voltage swings such as electrical muscle activity and the electrocardiogram (ECG), lower gains may be needed. On the other hand, very small signals recorded directly from the outside of nerve cells may require higher gain. Within this context one embodiment of the invention contemplates the simultaneous measurement of one or more signal types that may or may not be spatially distinct. Thus, the system provides a large range of user selectable gain control to allow flexibility in numerous applications.

In some embodiments, the system amplifies the signal with a small gain (e.g., ˜2.5) and no offset. At a second stage, the DC offset can be reduced and the gain can be increased (e.g., ˜10). At another stage, the DC offset can be subtracted and the gain can be increased again (e.g., the total gain can be approximately 50). In some embodiments, the system subtracts the DC offset and amplifies the signal with a gain. Other embodiments may include different stages and/or other processes.

Removing the DC offset of the neural signal can be a challenge while maintaining low noise, low power, low area, good power supply rejection, high linearity and SFDR performance. Typical neural signals from small animals have a DC offset up to +/−0.3 V due to a difference in potential between the ground connection on the skull and the neural sensors in the brain. Most neural-sensor designs use AC coupling to remove the DC offset but this requires large off-chip capacitors to meet the bandwidth requirement of 0.1 Hz. Several alternative DC offset calibration designs were investigated to determine performance.

The first design that injected current into the feedback path of the op-amp to cancel the DC offset was found to inject too much noise into the channel. This design requires a very low noise current source, with output noise below 1.1 μV, at least 4 times the die area and 20% more power consumption to reach the same performance as the current design.

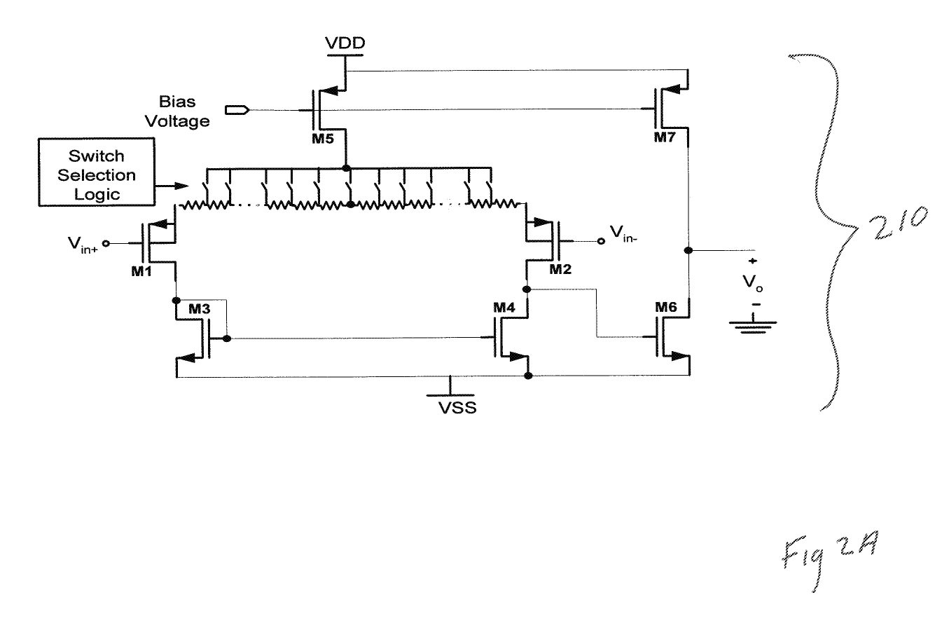

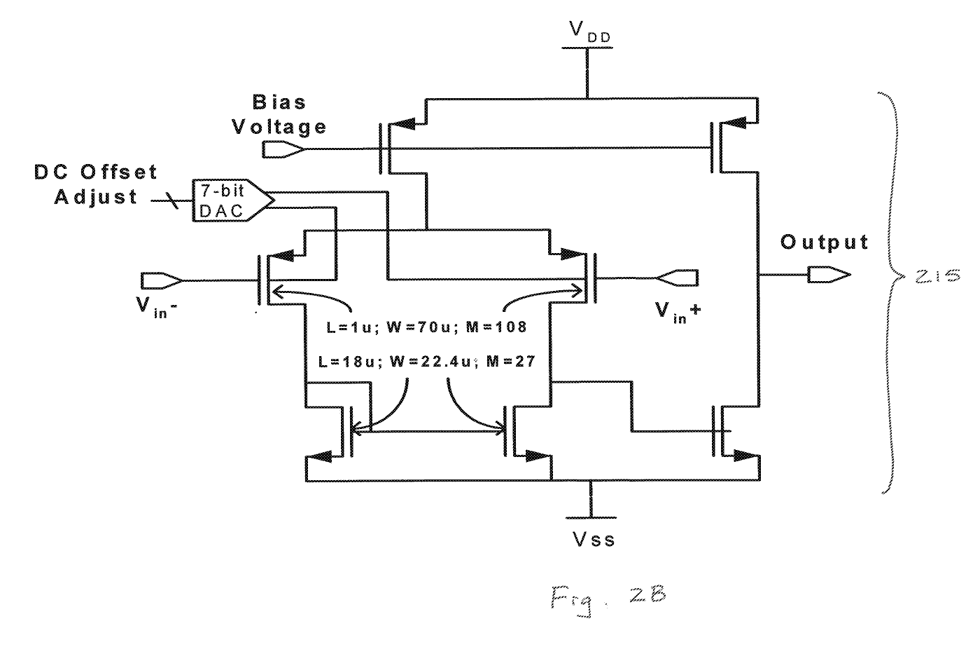

In some embodiments, a design that made the source degeneration resistors in the differential input pair transistors of op-amp asymmetrical to cancel the offset had low power, low noise, suitable power supply rejection ratio but poor SFDR. A schematic 210 is shown as FIG. 2A. With no DC offset the current from M5 is switched to the center point of the differential pair resistor string. With a non-zero DC offset the current from M5 is switched to resistor nodes either toward Vin+ or Vin− if the DC offset is positive or negative respectively. The voltage drop across the different resistances to the sources of M1 and M2 makes the source voltages at M1 and M2 different, canceling the DC offset. This design has an SFDR that is about 20 db lower than the current design.

The third design 215 that controls the bulk voltage of the differential input pair transistors of an op-amp to cancel the DC offset, shown in FIG. 2B, had low noise, low power, high SFDR and high gain. The DAC needs to have low noise since g_{mb} is only a factor of 4 or 5 lower than gₘ.

### Chopping

Chopping amplifiers can be used to reduce 1/f noise. FIG. 2C shows chopping applied to a two-stage operational amplifier 220 with body bias used to cancel the dc offset. The clock C and opposite phase of the clock NC control switches to modulate the signal. The basis of chopping amplifier is to modulate the signal to a higher frequency. When the signal is demodulated back to the signal band, the 1/f noise is moved to a higher frequency than signals of interest and then removed using a low pass filter. Chopping is performed at the drains of the input transistors and not at the input to keep the input current low. The input transistors have large input capacitance and switching would draw too large of an average current from the animal.

### Sampling

Simultaneous sampling of N multiple channels and holding each level until the ADC has completed the conversion of all N channels has the advantage that the time correlation between channels is highly accurate. However the sampling time is shortened compared to staggering the sampling of each channel. The output must settle to high accuracy during the sample time. A shorter time means a larger bandwidth which has more noise. Increasing the power of the sample and hold will lower the noise but higher power is also undesirable. Staggering the sampling time results in a lower noise power product, regardless of whether the sampled channel data is not aligned. If the bandwidth of the signal is below the Nyquist rate of the ADC then no information is lost and the waveform can be accurately reconstructed. Interpolation can be used after acquisition and transmission of data to time align the data samples. Data can be stored before or after interpolation.

The fourth design, which uses an instrumentation amplifier, required twice the power and layout area compared to using a single op-amp design.

In some embodiments, a solution to remove the DC offset with high accuracy low power and low noise is to cancel the DC offset in the second and third gain stages. Other solutions, however, may also provide suitable results.

The amplified signal is then filtered by a two pole Butterworth low pass filter set to 25% or lower of the typical digitizing speed per Nyquist frequency requirements. While the Nyquist requirements state that the low pass filters should be at least 50% of the digitizing frequency, we chose 25% to improve the noise specifications of the output and help prevent aliasing of the digitized data. However, other frequencies can be used.

Once the signal has been amplified and filtered, all of the channels can be sampled simultaneously by independent sample and hold circuits. Then, a multiplexer may serially transfer the data to an ADC. Most digitizing systems with multiple channels have one sample and hold circuit which serially samples the different channels, with the multiplexer before the sample and hold. One advantage of this technique is that the system may require only one sample and hold circuit. However, when time varying events occur across all of the channels, and the user wishes to correlate events across the channels, the actual time of the sample may be taken into consideration. When all of the channels are sampled at the same time, then correlation of waveforms across the channels is much more robust and mathematically simpler to compute with fewer assumptions about the stability of the signals over the time of the sample.

### Interleaving Architecture of the ADC

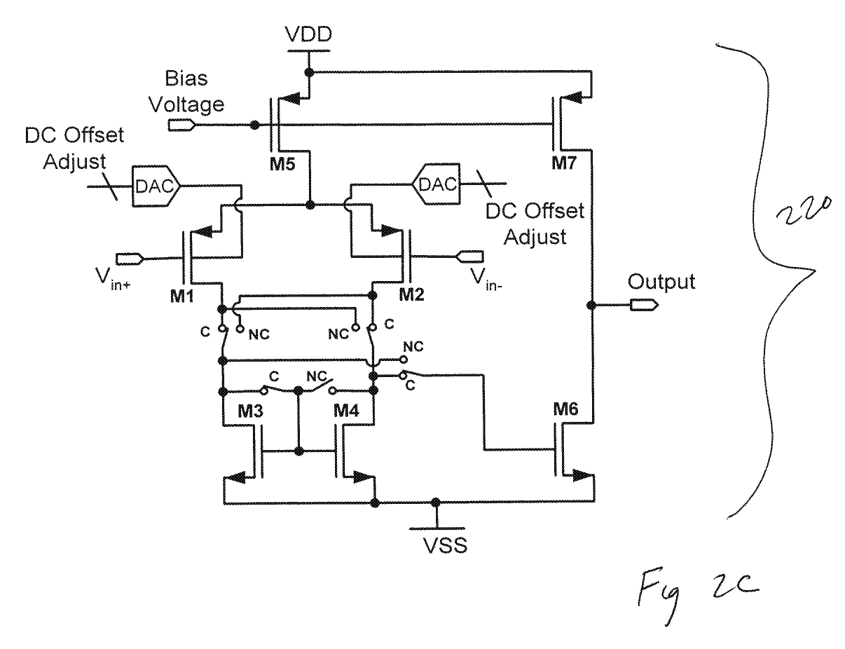

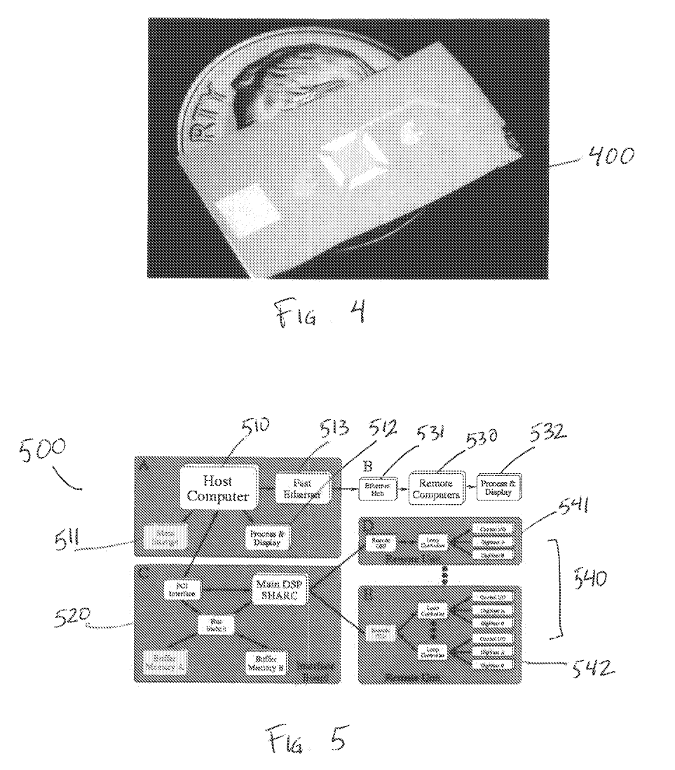

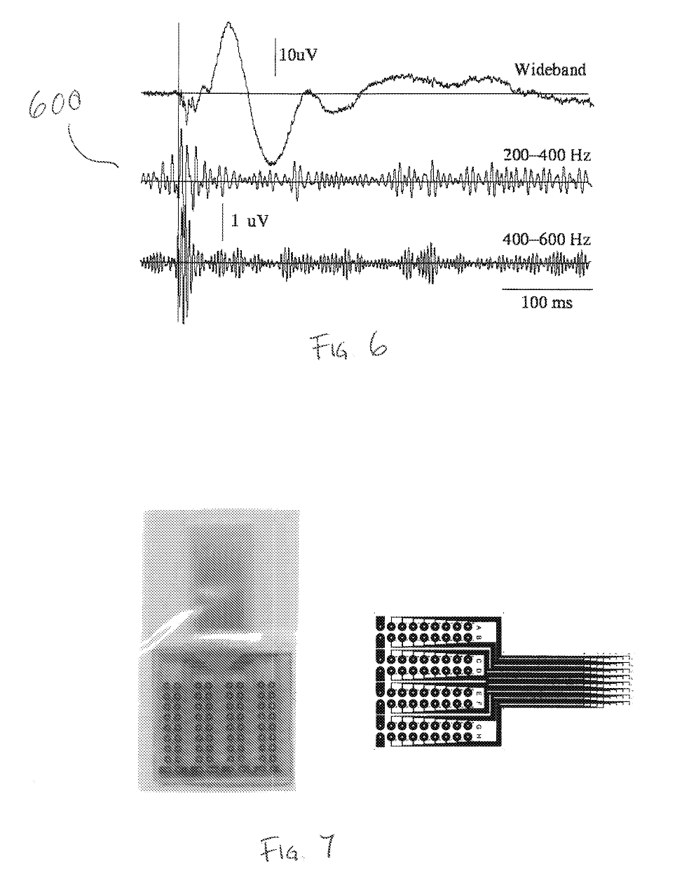

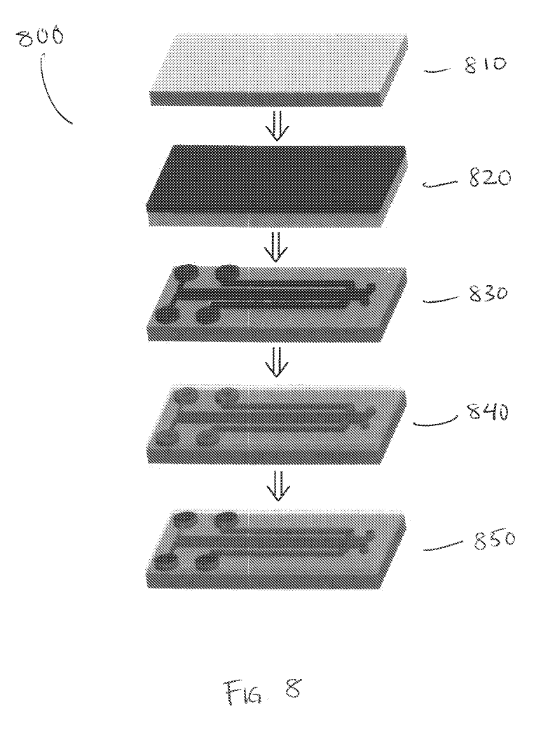

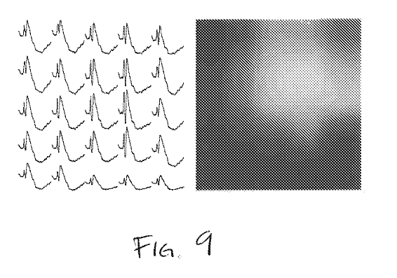

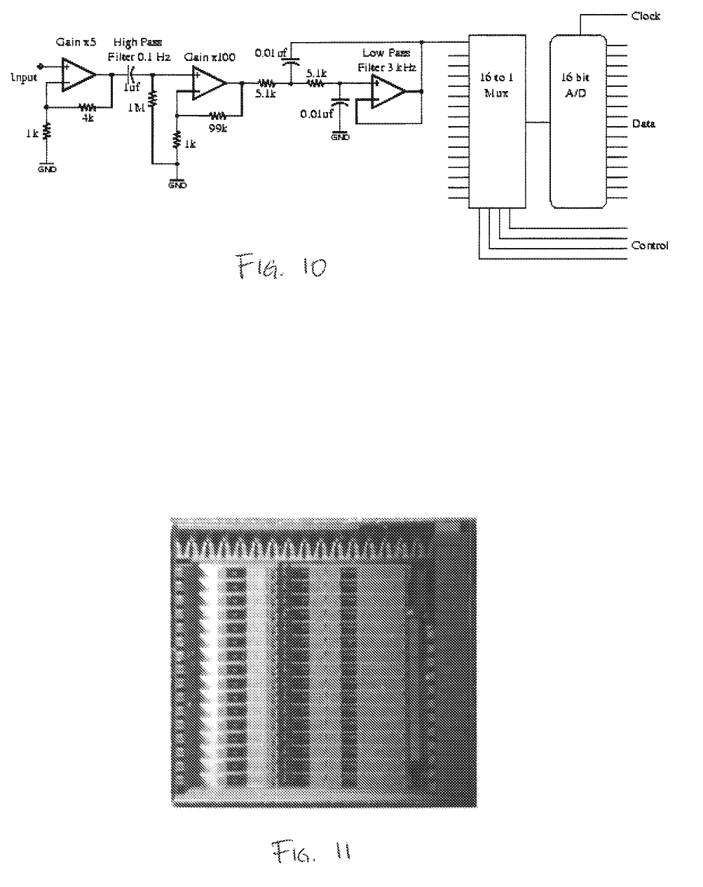

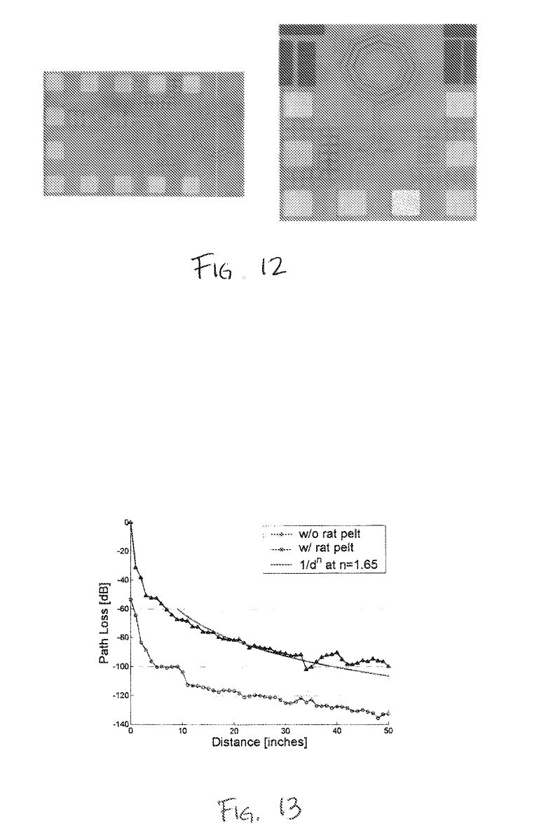

The ADC utilized in some embodiments herein includes an interleaving architecture for reducing power dissipation. The block diagram for an ADC 300 is shown in FIG. 3. The ADC architecture 300 can employ an interleaving method and an comparator design which contributes to reduced power consumption while maintaining accuracy and speed. The system involves a power reduction method for ADCs including two binary-weighted capacitor arrays, switches, comparators and control logic. In one embodiment, the system enables the power dissipation of successive approximation ADCs to be reduced by nearly a factor of two, and can be implemented as a means to amplify, filter and sample analog signals.

The ADC converter can include two binary-weighted capacitor arrays, switches, comparators and control logic. Two sets of capacitor arrays are used, wherein the op-amp buffer charges one capacitor array to the input voltage while the comparators are in the bit cycling mode on the other capacitor array. The time for the comparators to cycle through the bits and for the input buffer to charge the array is doubled. The output current requirements for the buffers drop to half that compared to having a single array and therefore the buffer power dissipation can also be reduced by nearly half. The comparators have twice the time to resolve voltage differences and the comparator power can also be reduced by nearly a factor of two.

The system operates as follows. During sampling mode, the upper capacitor array top plates are tied to the mid point of the power supplies (GND), while the lower capacitor top plates are tied to the analog input, and both of the capacitor array bottom plates are tied to the average voltage of analog input and GND. After the voltage settles, the bottom plates are disconnected from the voltage source and the charge remains on the bottom plates during the remainder of the conversion. During bit cycling the bits are determined successively in order from most significant bit (MSB) to least significant bit (LSB) by switching the binary-weighted capacitor top plates of the array to the reference voltages REF+ and REF−. The comparator determines which input is larger and the logic determines whether the switches remains set or not.

A self-calibration scheme may be used, which can eliminate common-mode noise as well as errors due to linear capacitor voltage-dependence. A new calibration technique was used to remove gain error by equalizing the values of the upper and lower bottom-plate parasitic capacitances. A dual-comparator approach was implemented with offset cancellation techniques to eliminate comparator offset.

Power dissipation is reduced by nearly a factor of two, while ADC throughput is maintained. Latency is increased by a clock cycle, but this is not a significant issue for many applications. The area of the capacitor arrays doubles, which will lead to a larger IC and therefore increase the cost of the interleaved ADC.

Although the ADC block diagram is used as an example embodiment of this component, the ADC is not limited by that system, many variants on the basic design elements are available to one skilled in the art.

In some implementations the data is transferred through a wire or wires that connect the detection element-signal processor to the data logger. Other embodiments may include alternative hard-wired configurations wherein the detection element-signal processor component is wired to a distant wireless transmitter. In one embodiment, the detection element is wired to the signal processor-wireless transmitter that is contained on a single chip. However, standard off-the-shelf wireless transmission methods may be utilized for lower data rate applications. In such implementations the signal processor may or may not be housed on the same chip as the wireless transmitter.

In one embodiment, wherein wireless transmission is utilized; a frequency generator produces an ISM band which is then modulated with On-Off Keying (OOK) to generate a carrier wave that can then be transmitted utilizing an RF antenna. To meet the size and power requirements, the telemetry system includes a very short-range communications link. Longer range communications may be suitable in other embodiments. For example, in some implementations wherein power requirements are not a major concern one may implement a transmission scheme capable of longer range communications. The wireless link in one embodiment may have a high data rate and ultra low power consumption. In one embodiment described herein the wireless communication link is implemented by employing Multi-Carrier On-Off Keying (MC-OOK) modulation that is driven by an associated base-band signal, such as frequency shift keyed (FSK) modulation or pulse position modulation (PPM). Alternative methods for the high data rate including but not limited to Multiple-Input Multiple-Output (MIMO) and Orthogonal Frequency Division Multiplexing (OFDM) may just as easily be implemented if appropriate for the demands of the device. This wireless link can effectively provide ultra low power, high data rate and short range radio communications for the bio-medical sensor applications.

The wireless communication link in one embodiment has low power and high data rate transmitter only to minimize power consumption. A simple low data rate and low power AM receiver is implemented through an inductive coupling scheme to transfer control data from the central sensor station (Main Computer) to the sensor chip. The wireless link uses Multi Carrier On-Off-Keying (OOK) method with either frequency shift keying (FSK) or pulse position modulation (PPM). The transmitter uses only one low phase noise local oscillator with dividers for multiple RF carrier signals which fall in 2 GHz and 5 GHz Industry, Science, Medical (ISM) frequency band. The divider for multi-carrier RF signal generation in the transmitter is implemented using digital logics with analog circuits instead of using power-hungry all digital circuit implementation to minimize total power consumption.

The link transmits multi-carrier RF signals modulated with a baseband signal as on-off keying (OOK) and either frequency shift keyed (FSK) or pulse position modulation (PPM) to achieve high data rate maintaining minimal power consumption and high power efficiency of the communication link. The channel efficiency may be compromised to increase the power efficiency for the target high data rate and ultra low power transmission. To achieve low power OOK, the transmitter incorporates CMOS Single Pole Single Throw (SPST) modulator. The RF carrier signal is implemented using an ultra low power low phase noise Voltage Controlled Oscillator (VCO) to accommodate multi-carrier OOK communication using one multi-band band VCO.

Multi-Carrier OOK RF pulses produced by the transmitter are encoded with the data values of which are represented by phase/frequency information, such as frequency shift keyed (FSK) data and pulse position modulated (PPM) data. In FSK implementation, the output of transmitter is keyed on and off by pulses at two different frequencies, respectively associated with different logical states of data. The Multi-Carrier OOK-FSK transmitter contains digital circuits for FSK encoded serial data.

The overall receiver in the central station (Main computer) is the same for the Multi-carrier OOK-FSK and Multi-Carrier OOK-PPM implementation. The receiver detects incoming data by detecting incoming Multi-Carrier OOK pulses using band pass filter. The common portion of each FSK and PPM receiver includes a multi-band antenna to provide RF selectivity and reject interference. The output of antenna and band pass filter is coupled to each AM detector with a low pass filter and can recover the weak power pulse train that was transmitted by the Multi-Carrier OOK transmitter. This pulse train can be compared with a comparator to recover all digital signals. In the OOK-FSK receiver, a FM discriminator circuit demodulates the FSK encoded data from the output of the comparator. The output of the FM discriminator varies between two voltages which are converted to respective logic levels.

In a Multi-Carrier OOK-PPM, the output of the transmitter is keyed on and off by pulses that are shifted in phase by the data. Data is encoded by shifting the phase. The digital portion of the OOK-PPM transmitter circuit comprises an arrangement of counters and shift registers to obtain parallel data and then converts this data into the OOK modulation pulses representing the encoded serial data. The receiver may add over sampling circuit to the recovered pulse train.

As an alternative to significantly reduce the power consumption of the sensor node maintaining high data rate the ultra-wideband technology can be used. Ultra-wideband offers a possibility for ultra-low power designs, by utilizing short impulses with very low duty cycles for communications. This significantly reduces the average power of the wireless communication link. With a power management, ultra low power and high data rate sensor nodes can be realized.

The UWB transmitter can include three main blocks: 1) a modulator which encodes the input binary data with an external clock; 2) a pulse generator which outputs sub-nanosecond impulses; and 3) a 50Ω driver for the antenna. A frequency synthesis technique, based on a delay locked loop (DLL) and a digital edge combiner, may be used in the RF clock generator.

Pulse generator is one building block in an UWB impulse radio system. This is to generate sub-nanosecond Gaussian pulses in the time-domain for data transmission. Due to the exponential relationship in the Gaussian pulse, a bipolar transistor can implement this function. However, from integration and cost point of views, a CMOS technology node is used to implement the pulse generator. The main challenge is to use a simple and power efficient technique to approximate Gaussian monocycles in CMOS, which meet the FCC spectral mask requirements.

A pulse shaping network is implemented to meet the FCC spectrum mask limit at lower frequencies. The driver circuit often consumes more than 50% of the total transmitter power. By employing a co-design technique of the driver stage with the pulse shaping network, significant power saving is achieved. The design essentially acts as a switching amplifier where power is only consumed when there is a transmitting pulse from the pulse generation. Hence, with a low transmission duty cycle, a low average power consumption can be achieved. To minimize the power consumption of the modulator block, modulation is co-designed with the system clock. Two separate modulation schemes, namely OOK and PPM, can be designed.

This CMOS UWB pulse-based transmitter is targeted for ultra low power and high data rate and short range wireless communication link for the sensor system. In some embodiments wherein a long range data transmission is desirable implementations utilizing alternative strategies at the expense of power consumption are considered. In these implementations the detection element and amplifier chip may be located remotely at a position on a subject and hard wired to the transmitter. Alternatively, a detection element may be connected to an amplifier-transmitter. In one embodiment, short relays can be used wherein a low power amplifier-transmitter chip sends data to a more powerful transmitter located at a remote location on or near the subject, which in-turn transmits the data over a long distance.

Many potential methods are available to power the device, any of which may be a viable strategy depending on the intended use for the device and the specific power requirements. In applications where all of the components are placed external to a subject a battery powered unit would be sufficient, however, alternative solutions may be required in implementations wherein the components are positioned within a subject. Other important variables in the choice of power source related to the timescale over which one needs to collect data: Shorter experimental durations may require less energy.

The major limitation of batteries is that they hold a fixed amount of energy. Once that is expended, they may either be replaced or recharged. Lithium primary batteries for example, have a power density on the order of 100 W/kg and a stored energy density on the order of 100 Whr/kg. Thus a 1 g battery will supply the 100 mW required for these experiments. In scenarios wherein a short time scale is sufficient, batteries can be positioned on the surface or implanted in the abdomen of the subject with leads directed subcutaneously to the amplifier/transmitter chip. For longer lasting recordings, in-vivo charging methods and inductive power coupling may be utilized.

Batteries can be recharged in-vivo or an inductively coupled system can be used for power and RF telemetry. The typical inductive power system consists of a primary coil, a secondary coil, circuitry for impedance matching, and a power source. Power from a large external primary coil mounted directly beneath or around the animal enclosure can be magnetically coupled to a small implanted secondary coil. Two concerns with the drive frequency are the effect of attenuation in the tissue and the effect of the magnetic fields on the rest of the circuitry, however, a frequency of 2 MHz has been successfully used previously for biological applications.

Because the subject is free to move about, the orientation of the implanted secondary coil can change from fully horizontal to fully vertical. Since the primary coil has a fixed and stationary orientation, the coupling between the primary and secondary coils can vary from the maximum to zero. To capture data over most orientations two or three secondary coils that are perpendicular to each other may be used. Since flat and flexible coils may be utilized, several may be inserted either on and/or under the skin or the subject in different orientations. Additionally, RF signals can locate the subject position, and operate an array of overlapping coils around the cage to maximize power delivery.

Power supply that can harvest energy from the animal itself or the surroundings are also contemplated. Means to accomplish this include 1) heat engines to harvest energy available from temperature differences, 2) piezoelectric materials to harvest vibration energy, and 3) solar cells to harvest radiation (e.g., light). Piezoelectric materials may be implanted to harvest energy from the animal's movements (Hausler 1984). Alternatively, a MEMS based heat engine can use waste heat from the animal for electric power such as the heat engine 400 shown in FIG. 4.

Since the power requirements of the A/D chip and RF telemetry may be high, no single power source listed above may be able supply the amount of electric power required. Thus, in some embodiments, hybrid systems that utilize two or more of the above concepts can be combined to supply the requisite power.

A computing system 500 that can simultaneously acquire video and multiple analog signals at 12 and 16 bits resolution is utilized for the examples provided herein and is shown in FIG. 5. In one implementation, a computer 510 is in two-way communication with the device: This allows for real-time data acquisition and control of the gain setting for any individual channel within the detection element. In addition to real time display during acquisition, the system is multi-threaded to take advantage of a multi-processor environment and performs on-line analyses for time triggered averages, digital filtering and channel subtraction. To create animated sequences, the software performs sequential, time triggered subtraction, ratio, and calibration calculations. On-line analysis includes frame-by-frame standard deviation and FFT computations. The computer 510 can display intensity histograms, as well as raw or averaged images and dynamic average pixel intensity over the image or specified subregion along with a strip chart style display of physiological data.

The computer 510 may include components such as a storage components 511, processing and/or display components 512, data link components 513, and other typical components (such as inputs components, and so on). The computer 510 may communicate with other computers 530 and/or display components 532 via a wireless communication link 531, such as an ethernet hub. The current digitizing system is built on a PCI interface card 520 for high speed data transfers. The PCI card 520 transfers data to digitizers 541, 542 (collectively 540) over high speed links. The PCI card 520 contains 8 MB of double buffered memory for synchronous acquisition and download capability and uses 7 ns PLDs to execute the various control functions for digitizing and CCD control. This system has performed well for rapid and flexible video and electrophysiological acquisition. Data is stored in the on-board memory in a standard file format ‘IFFPHYS’, an internationally recognized format for physiological data. The PCI card plugs into any standard PC motherboard. Simple drivers written for the LINUX operating system control the card and download buffered data.

### EXAMPLES

The following are example implementations and/or configurations of various components of the system described herein. Of course, other example implementations and/or configurations are possible.

### Flexible Flat Electrodes as Detection Elements

A 64 channel flexible electrode array was fabricated in our facilities and later used with no damage to the underlying cortical tissue. We obtained excellent evoked responses from the cortical surface (shown as graph 600 in FIG. 6). In rodents, the dura is relatively thin, making these measurements even easier. Since the copper metal of commercially available flex-circuit boards may not be ideal for long term chronic recordings, and the trace configuration is not mapped in a 2-D array, we have developed our own flat and flexible electrode arrays using gold contacts arranged in a 2-D grid configuration (shown in FIG. 7).

Several capabilities for prototyping micro-electromechanical system (MEMS) devices have been integrated. To fulfill the requirements for measuring the electrical activity of the cortical surface, an array of finely spaced electrodes was deposited on a flexible substrate to fit underneath the rat's skull. The electrode array is an 8×8 or 5×5 grid, covering a 5×5 mm area constructed from three basic components; a Kapton substrate, a patterned electrical circuit, and a photoimagable film to protect and electrically insulate the leads where needed. All layers result in a total thickness of 75 um. This enables the realization of almost any size, pattern, or shape electrode array.

The process 800 is shown in FIG. 8. The circuit is constructed from patterned gold that connects the 64 electrodes to 64 contact pads which are protected by a photoimagable, chemically amplified, epoxy-based negative photoresist, SU-8 (Dupont, Wilmington Del.). SU-8 was chosen for its high aspect ratio imaging, thermal stability, and good chemical resistance, but other polyimides may be used. We first sputtered 810 a 5 nm layer of Titanium-Tungsten onto the Kapton substrate followed by a 300 nm gold layer 820 using DC Magnetron sputtering. The Titanium-Tungsten layer was used as an intermediary layer between the Kapton film and gold to promote adhesion. The gold is then sputtered onto the adhesion layer 830 and etched using photolithography to pattern an array of 50-100 um spots on a 150 to 500 um spacing. Photolithography was used to pattern the gold to produce the circuit which involved applying a thin layer of Hexamethyldisilazane followed by a layer of AX5214-EIR photoresist on the Kapton substrate using a spin coater and then baked on a hotplate. The Hexamethyidisilazane is an adhesion promoter layer between the photoresist and the gold. The patterning of the photoresist is performed by flooding the photoresist with ultraviolet light through a mask which changes the chemistry of the photoresist making it soluble for etching. The unwanted gold was removed using a gold etch bath. The Titanium-Tungsten was removed 840 by hydrogen peroxide bath in the same pattern as the gold. Finally, an acetone wash was used to remove 850 the photoresist leaving behind the completed gold circuit.

Photolithography was also used to pattern the SU-8. To prepare the Kapton and gold sample for SU-8 the sample was plasma etched, washed with acetone, isopropanol, and deionized water, then baked to remove all remaining organics. A 13 um layer of 2010 SU-8 photoresist was spun on top of the Kapton and gold circuit, then baked to crosslink the SU-8 fibers. The sample was again baked and the unwanted SU-8 was removed using SU-8 developer bath. The sample was finally washed with acetone, isopropanol, and deionized water, dried, and baked again to insure the thermal properties of the SU-8. We evaluated the electrical characteristics and integrity of the electrodes by measuring the impedance of each trace from electrode to connection pad in a 0.9% saline solution (100 kOhms±200) with at least an 90% overall yield.

Electrode arrays were implanted in 7 male Sprague-Dawley rats (400-500 g) for testing. Animals were anesthetized with Ketamine/Xylazine cocktail (100 mg/kg and 10 mg/kg respectively) and body temperature maintained at 37° C. We administered 20% of the initial dose as supplemental anesthesia when needed. We then placed the animal in a standard sterotaxic frame and drilled a 5×1 mm slit in the rostro-caudal direction between bregma and lambda just lateral to the midline. We beveled the midline edge of the craniotomy to assist in the insertion of the probe. Using a firm plastic knife inserted under the bone, we separated the dura from the skull to promote an electrode array insertion. The electrode array then slipped easily between the dura and the skull over the whisker barrels somatosensory cortex on the right side of the animal.

During a 4 hour recording the animal was introduced to 0.2 ms whisker twitches on the left side from a 5 volt signal generator to an 8 ohm speaker based whisker actuator attached to a 10 cm length of hypodermic tubing into which the whisker was placed. Each whisker twitch was randomized between 1 to 2 seconds. Surface field potentials recorded by the electrode array were collected with a 16 bit digitizer at 20 kHz and plotted, as shown in FIG. 9.

### Specifications of the SoC

In several applications, the specifications of the system IC are to amplify and filter at least 16 electrophysiological signals over a bandwidth from DC to 4 kHz, and then digitize and multiplex the data over one wire. The input impedance of the source ranges from 100 Kohms to 1 Mohms so that the input referred noise of the system should be less than 2 uV rms from 1 Hz to 7 kHz. The gain should be controllable over a range of 2 to 200 to accommodate different applications and not let large DC offsets of the input signal saturate the amplifiers. Usually, a high-pass filter is used to offset any DC bias on the inputs. We employ 13 bits of DC offset control that can track and adjust large voltage level swings to the digitizer at least at every 1 second interval. This procedure allows us to maintain DC levels throughout the system, eliminates the need for a high pass filter, and effectively increases the dynamic range of the digitizer. A 12-bit to 16-bit analog-to-digital converter (ADC) is may be used to provide enough resolution for each channel. An example preamplifier and digitizer system is shown in FIG. 10.

Developing the amplifiers and digitizer on separate chips to reduce the risk of a non-functional device since existing components can be piggy-backed onto the amplifier. A combination of the MAX 396 and LTC1608 used in our current systems already represent off-the-shelf components that would multiplex 16 channels and provide 16 bits resolution at 500 kHz in a small package.

Direct coupling of the signal inputs carries some advantages. The current amplifier design uses 7 offset bits to adjust DC levels before amplification, enabling high gain for the first stage. Since most of the DC offset comes from electro-chemical potentials in the electrodes, we may not need to adjust the offset very often. Capacitive coupling the inputs may eliminate any potential problems with the offset control, and reduce the requirements of the digitizer to 12 bits.

DC coupled inputs allow sampling across a very wide band of potential signal sources, and enable digital filtering techniques to be used to parse the data stream for a variety of relevant information. Substantial and potentially drifting input offset due to electrochemical potentials can be assessed with DC coupled amplifiers. Cortical EEG contains DC and slowly varying (<0.01 Hz) components which assist in assessing tonic excitation of apical dendrites and cortical pyramidal neurons. Additionally, DC signals such as temperature can be recorded without FM modulation. By supplying a sine wave input of known amplitude, it is possible to generate a lookup table to correct for inaccuracies in the offset D/A.

### SoC Prototype Design and Simulations

We have designed and tested a 16-channel prototype using the MOSIS service and TSMC's 0.25 micron CMOS process that nearly meets the above specifications. The prototype operates with +/−1.5V power supplies and dissipates about 10 mW (˜0.7 mW per channel). A controller on the IC receives incoming commands and sets the gains of each channel, controls the offset voltage correction circuitry and controls the data flow of serial transceiver.

A photograph of the completed chip is shown in FIG. 11. The first amplifier has a gain of 10 and any DC offset is canceled by the first stage offset control so the output will not saturate the input of the second stage. This second amplifier is a rail-to-rail operational amplifier that has selectable gain of 1 or 10, also with a selectable offset control. The third amplifier has selectable gains of 1, 3 and 10. This provides selectable overall gains of 10 to 1000 and 13 bits of offset control. The integrated root-mean squared (rms) input referred noise over a bandwidth of 1 Hz to 7 kHz is 1.9 uV. Very large devices were used to reduce the 1/f noise at low frequencies to achieve this low noise. The rms noise associated with input impedances of 100 Kohm and 1 Mohm are increased only slightly from 3.4 uV and 10.8 uV to 4.2 uV and 11.0 uV, respectively, by the relatively low noise of the amplifiers.

### SoC 16 Bit 500 kHz Digitizer

Fourth-order Butterworth filters are used to limit the bandwidth before the delta-sigma ADCs so that aliasing does not occur. This may include a low-power 6.2 mW 16-bit 500 k samples per second (Sps) analog-to-digital converter (ADC). We chose a charge-redistribution self-calibration successive approximation ADC approach due to its low power consumption, high accuracy and relatively high speed. Because there are multiple channels to be digitized, delta-sigma ADC approaches result in higher power dissipation. The most suitable commercially available ADC, the AD7694, provides 15 bit accuracy up to 100 kSps with about 2 mW power dissipation and a 3 V power supply. The AD7694 does not include the input amplifier which dissipates several mW. Our ADC has a conversion rate about 5 times higher for only about twice the power dissipation of 3.9 mW (after subtracting the input buffer power dissipation of 2.3 mW.) In addition, the accuracy is 1 bit higher. The key reasons for the performance improvement are the comparator design and the use of two sets of capacitor arrays to reduce power by interleaving. These outputs are multiplexed and output serially at a data rate of 8 Mbps.

### Wireless Telemetry

We have designed low power transmitter systems for this application. For RF front-end transmitters, we have developed Industrial, Science, and Medical (ISM) band radios. The front-end was implemented for 5˜6 GHz ISM band applications, similar to our frequency range. (e.g., the single chip OOK (On-Off-Keying) wireless transmitter shown in FIG. 12). The transmitter consists of carrier signal generator, namely voltage controlled oscillator (VCO), SPDT (Single Pole Double Throw) switch as a modulator and an RF antenna. A drive amplifier can be added if higher transmit power is required.

In this application, serialized data from the ADC can drive the modulator. A fast and high isolation SPDT switch designed to accommodate high data rate and low noise during transmission is implemented. The signal to noise ratio (SNR) at a given distance depends on the quality of the signal carrier (VCO), performance of the switch and the power level at the RF antenna. In one approach, modulated signals are transmitted through ISM band channels in the 5˜6 GHz range where transmitted power is inversely proportional to the square or some fraction (n) of the distance between the RF receiver and the transmitter. Channel impairments are caused by white Gaussian noise, multi-path fading effects that introduce intersymbol interference (ISI) and fading effects. At high carrier frequency the communication is generally point to point, and therefore, it is possible to achieve high data rates with reasonable SNR. Based on Shannon's law (Shannon, 1948), short range wireless communication allows a 5˜6 GHz ISM band radio up to the targeted data rates. The throughput of this approach can be more than 50 Mbps due to the target short reach communication and may exhibit better noise and power specifications than wide-band transmitters. In other designs, a multi-carrier 5˜6 GHz ISM band radio using a digital modulation scheme can be used. In addition, other high data rate wireless communication methods such as multi-input and multi-output (MIMO) and Ultra Wideband (UWB) wireless link may be used.

FIG. 13 shows the measured channel path loss at 5.6 GHz frequency between transmitter and receiver. Channel characterization data in (Kivinen et al., 2001) also supports the use of 5˜6 GHz frequency range for data transmission. The path loss coefficient (n) in the line of sight (LOS) for our measurement is in range n˜1.65 which correlates well with (Kivinen et al., 2001) where n˜1.4 for 5.3 GHz. When a simple quarter wave filament RF antenna was embedded inside a rat pelt and fat layer, the path loss is increased by roughly 30 dB as shown in FIG. 13. The channel loss can be easily reduced by using an improved high gain antenna and driver amplifier in the transmitter, as in (Kivinen et al., 2001), to extend the distance up to 1-2 meters which may be sufficient for the target application. Additionally, a high sensitivity RF receiver can further mitigate these losses.

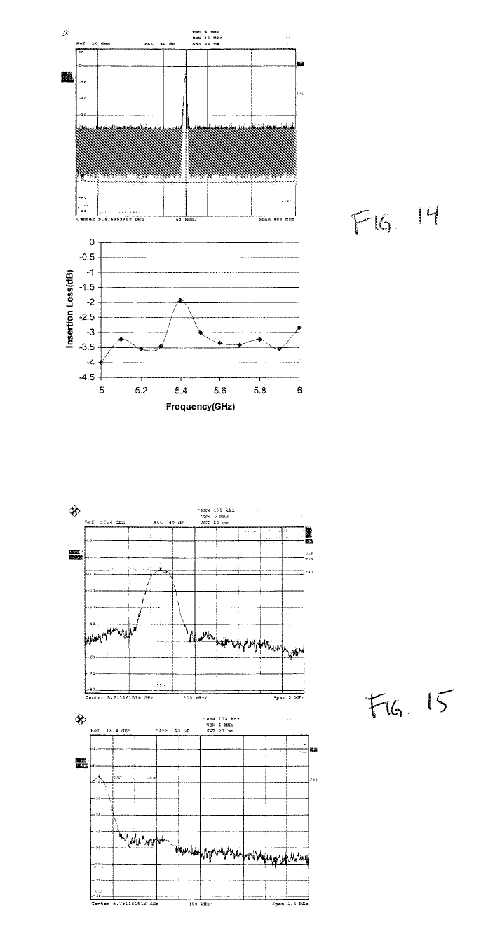

FIG. 12 shows the chip photos and FIGS. 14-15 illustrate the measurement data for the fabricated prototype OOK transmitter in 0.25-um CMOS technology utilizing 5˜6 GHz ISM band. A low power complementary cross-coupled LC voltage controlled oscillator is used for carrier generation. The VCO consumes only 5 mW power to achieve the measured output power of −5 dBm (FIG. 14) and single-sideband phase noise of −92.75 dBc/Hz and −97.6 dBc/Hz at 600 KHz and 1.0 MHz offset frequency, respectively (FIG. 15). The phase noise of the VCO is a factor in achieving high data rate since high phase noise results in dispersion of noise from one channel to adjacent channels, resulting in lower SNR and higher data error rate. The fabricated LC VCO meets the target phase noise requirements sufficiently and requires less than 0.7 mm² silicon real estate.

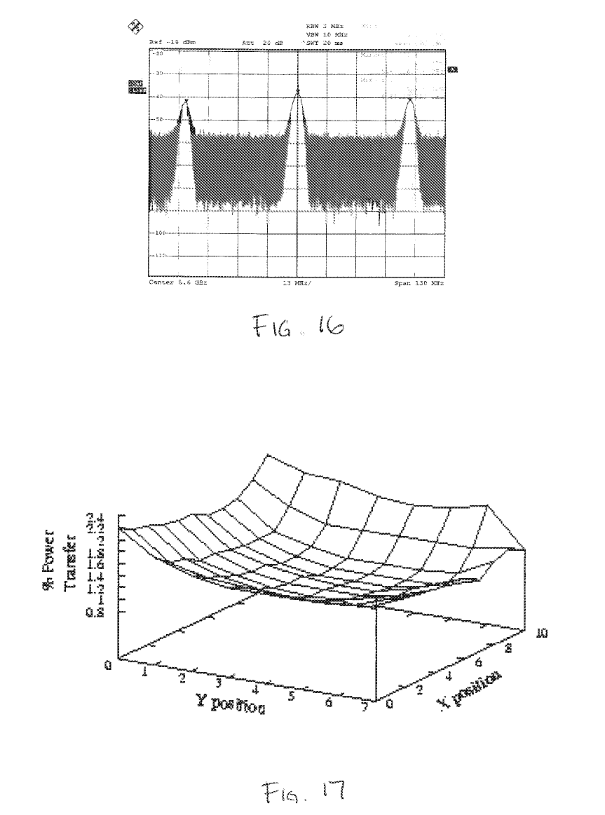

The modulator, SPDT switch, for the On-Off Shift Keying shows insertion loss (IL) of 3.3 dB at 5.6 GHz (FIG. 14). The fabricated RF modulator may require less than 0.6 mm² silicon real estate. The initial targeted data rate is 10 Mbits/s and measured data rate of the integrated OOK transmitter system (FIG. 10) shows that this target can be surpassed comfortably. FIG. 16 shows the transmit spectrum of OOK system at 100 Mbits/s modulated with digital 1010 data pattern. The measurements show the capability for use of such system in the design with use in multi-carrier transmitter targeting up to 256 channels and 150 Mbits/s. The OOK transmitted data can be demodulated using a RF receiver with high input sensitivity. We have developed an RF front-end for the 5.6 GHz ISM band direct conversion receiver. A new direct-down receiver may also be used to improve input sensitivity, power dissipation and overall size for the wireless link. For the wireless link design, the target silicon size for the receiver and transmitter is less than 1.5 mm×1.5 mm and in addition, the power consumption is less than 10 mW. The target size and power consumption could be 1 mm×1 mm and 20 mW, respectively. A design of a multi-carrier 5˜6 GHz ISM band transceiver incorporating baseband analog circuits may also be included. This technique is utilized to implement a high data rate, compact, low voltage, and low power RF transmitter and receiver for the wireless link for the telemetry system. Other cutting-edge technologies such as Ultra Wide Band (UWB) and Multi-Input and Multi-Output (MIMO) may also be implemented within this application.

### Inductive Power

Inductively coupled power sources using a 20W power amplifier with transmitting coils either under the cage, or wrapped around the cage can be used. Miniature 10 mm coils acting as the receivers provide power with at least a 1.5% efficiency over the area of a 8 by 10 inch cage (FIG. 18). We have also had good results with remote powering using a 1 cm flat coil with a 100 mm square primary coil and obtained 140 mW output after rectification with 10 W input power. The bottom of the cage can be tiled with overlapped flat coils and switch power to them as the subject moves. Should power delivery prove problematic, data can be transmitted via the RF link to track the animal's position. Maximizing the area of the pickup coil may improve inductive powering. Flat receiver coils built on flexible bio-compatible substrates, such as Kapton, have the advantage that they can be inserted under the skin for maximum area.

Of particular concern for inductive power solutions is the attenuation experienced by the skin. For most of our applications, the telemetry device can be mounted on top of the animal's head after the scalp has been removed, however, there are many situations when the device may be implanted subcutaneously. Though it is clear that such attenuation occurs, it is unclear how this attenuation will affect the efficiency, especially for short distance.

### Data Acquisition System

We built a custom system that acquires video and multiple analog signals simultaneously at 12 and 16 bits resolution. A computer system displays intensity histograms, as well as raw or averaged images and dynamic average pixel intensity over the image or specified subregion along with a strip chart style display of physiological data.

The current digitizing system is built on a PCI interface card for high speed data transfers. It contains 8 MB of double buffered memory for synchronous acquisition and download capability and uses 7 ns PLDs to execute the various control functions for digitizing and CCD control (FIG. 5). This system has performed well for rapid and flexible video and electrophysiological acquisition. Data is stored in the on-board memory in a standard file format ‘IFFPHYS’, an internationally recognized format for physiological data. The PCI card plugs into any standard PC motherboard. Simple drivers written for the LINUX operating system control the card and download buffered data.

In addition to real time display during acquisition, a custom data acquisition software system can be multi-threaded to take advantage of a multi-processor environment and performs on-line analyses for time triggered averages, digital filtering and channel subtraction. To create animated sequences, the software performs sequential, time triggered subtraction, ratio, and calibration calculations. On-line analysis includes frame-by-frame standard deviation and FFT computations.

Software for parsing the physiological record ‘PARSENDP’ (FIG. 21) was written in ‘C’ under the Linux operating system using the ‘Gnome Toolkit’ (GTK, http://www.gtk.org) as the user interface using a package ‘GLADE’ (http://glade.gnome.org) to easily implement windows, buttons and widgets. GTK and GLADE are freely available under the GNU Public License, and are included in most Linux distributions. Many of the procedures and algorithms described here were initially tested using ‘OCTAVE’ (http://www.octave.org), a freely available mathematical and data analysis tool compatible with MATLAB (http://www.mathworks.com). The Linux operating system offers a number of advantages over other systems including high performance in a multi-tasking environment, easy access to free code for high performance applications, and compatible with low cost hardware.

PARSENDP is designed to read data organized with “headers” and “chunks” such as IFFPHYS. As long as the file provides the number of channels and sample rate, a function can be written for PARSENDP to read the data. Initially, a window is opened containing the physiological traces plotted across 10 s of time. Each channel can then be individually selected or deselected for viewing with different gain and IIRC Butterworth digital filtering specifications. A scroll bar allows the user to scan through each page of the file.

A button on the PARSENDP window will open another window for viewing sleep scoring analysis. After selecting the frequency resolution and epoch size, each channel in the record for each epoch is converted into the frequency domain using FFT procedures. The spectral data is then binned into total power for selected frequency ranges to determine sleep state: Delta (0.1-3 Hz), Theta (3-8 Hz), Alpha (8-12 Hz), Sigma (12-16 Hz), Beta (16-25 Hz), Gamma 1 (25-35 Hz), and Gamma 2 (35-45 Hz).

### CONCLUSION

Unless the context clearly requires otherwise, throughout the description and the examples, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. When the disclosure uses the word “or” in reference to a list of two or more items, that word covers all of the following interpretations of the word: any of the items in the list, all of the items in the list, and any combination of the items in the list.

The detailed descriptions of embodiments of the invention are not intended to be exhaustive or to limit the invention to the precise form disclosed above. While specific embodiments of, and examples for, the invention are described for illustrative purposes, various equivalent modifications are possible within the scope of the invention, as those skilled in the relevant art will recognize. For example, while steps are presented in a given order, alternative embodiments may perform routines having steps in a different order. The teachings of the invention provided herein can be applied to other systems, not necessarily the system described herein. These and other changes can be made to the invention in light of the detailed description. Moreover, the elements and acts of the various embodiments described herein can be combined to provide further embodiments.

These and other changes can be made to the invention in light of the detailed description. In general, the terms used in the following disclosure should not be construed to limit the invention to the specific embodiments disclosed in the specification, unless the above detailed description explicitly defines such terms. Accordingly, the actual scope of the invention encompasses the disclosed embodiments and all equivalent ways of practicing or implementing the invention.

1. A method for measuring a physiological parameter of a mammalian body, the method comprising:
sampling the physiological parameter with a plurality of contacts positioned at the body to determine two or more sampled values for the physiological parameter;
constructing a value for the physiological parameter using the two or more sampled values; and
adjusting for a DC offset.

2. The method of claim 1, wherein adjusting for a DC offset comprises applying an offset voltage to a signal corresponding to a sampled value of the physiological parameter.

3. The method of claim 1, further comprising converting the signal from an analog signal to a digital signal.

4. The method of claim 3, further comprising wirelessly transmitting the digital signal to a data station.

5. The method of claim 1 wherein applying an offset voltage comprises applying a dynamic offset voltage.

6. The method of claim 1, further comprising applying an electrical stimulus to the body via one or more of the contacts.

7. The method of claim 1, further comprising implanting the contacts within the body.

8. The method of claim 1, further comprising placing the contacts at a skin of the body.

9. The method of claim 1, further comprising positioning the contacts at a cortical surface of the body.

10. The method of claim 1 wherein sampling the physiological parameter comprises sampling an electrical potential of the body.

11. The method of claim 1 wherein sampling the physiological parameter with the contacts comprises sensing the physiological parameter with a plurality of detectors arranged in an array.

12. The method of claim 1 wherein:
sampling the physiological parameter with the contacts comprises at least approximately simultaneously sampling the physiological parameter with first and second contacts; and
the method further comprises at least approximately simultaneously sampling the physiological parameter with third and fourth contacts positioned at the body.

13. The method of claim 1 wherein:
sampling the physiological parameter with the contacts comprises sampling the physiological parameter with first and second contacts at a first time interval; and
the method further comprises sampling the physiological parameter with third and fourth contacts at a second time interval different than the first time interval.

14. An apparatus for measuring a physiological parameter of a body, the apparatus comprising:
an array of contacts for sampling a physiological parameter of a body; and
a processing device electrically coupled to the contacts to receive signals from the contacts, the processing device including a voltage offset component for adjusting for a DC offset, an analog to digital converter for converting the signals from analog signals to digital signals, and an output component for transmitting the digital signals to an external device.

15. The apparatus of claim 14 wherein the voltage offset component, the analog to digital converter, and the output component are disposed on a single chip.

16. The apparatus of claim 14 wherein the output component comprises a wireless transmitter.

17. The apparatus of claim 14 wherein the array of contacts and the processing device are sized for implantation into the body.

18. The apparatus of claim 15 wherein the voltage offset component is configured to apply a dynamic offset voltage to the signals.

19. A system for measuring a physiological parameter of a body, the system comprising:
a signal reception component that receives a signal related to a value of the physiological parameter measured from a contact at the body; and
a modulation component that modulates the signal to a higher frequency in order to reduce noise within the signal.

20. The system of claim 19, further comprising:
a demodulation component that demodulates the modulated signal; and
a filter component that removes frequencies of the demodulated signal that include the noise.

21. A system for measuring a physiological parameter of a body, the system comprising:
a signal reception component that receives signals from contacts at the body used to sample values of the physiological parameter, wherein each value is sampled at a time different than the other sampled values
a sampling component that reconstructs a value for the physiological parameter using the sampled values; and
a DC offset component that adjusts the value for a DC offset.

22. The system of claim 21, wherein reconstructing the value for the physiological parameter includes using interpolation to align the sampled values in timed order to construct a waveform based on the measured physiological parameter.

23. A method of measuring a value of a physiological parameter from a mammalian body, the method comprising:
sampling from an array of contacts at the mammalian body at frequencies of at least 7 kHz;
compensating for a DC offset from a signal substantially less than the DC offset to achieve high gain; and
reducing power consumption by chopping the signal.

24. The method of claim 23, wherein the array of contacts covers a 5 mm by 5 mm area of the mammalian body.

25. The method of claim 23, wherein the array of contacts is 5 mm by 5 mm in size.

26. The method of claim 23, wherein the array of contacts is 8 mm by 8 mm in size.

27. The method of claim 23, wherein sampling at frequencies of at least 7 kHz includes sampling at frequencies between 7 kHz and 10 kHz.

28. The method of claim 23, wherein sampling from an array of contacts includes serially sampling at least two contacts within the array of contacts.

29. A system for measuring a value of a physiological parameter from a mammalian body, comprising:
a sampling component that samples an array of contacts at the mammalian body at frequencies of at least 7 kHz;
a signal compensation component that compensates for DC offset of a signal received from the sampling component; and
a power reduction component that chops a portion of the received signal after the DC offset compensation.

30. The system of claim 29, wherein the signal compensation component applies an offset voltage to the received signal.

31. (canceled)

32. The system of claim 29, wherein the sampling component samples the array of contacts below the Nyquist rate and constructs the received signal.

33. The system of claim 29, wherein the sampling component samples the array of contacts serially.

34. The system of claim 29, wherein the power reduction component modulates the received signal to a higher frequency and demodulates the received signal to remove noise from the received signal.
